(19) United States
(12) Patent Application Publication
(10) Pub. No.: US 2023/0193245 A1
(43) Pub. Date: Jun. 22, 2023

(54) METHODS AND COMPOSITIONS FOR MAKING AND USING PEPTIDE ARRAYS

(71) Applicant: Google LLC, Mountain View, CA (US)

(72) Inventors: Asmamaw Wassie, Boston, MA (US); Annalisa Marie Pawlosky, Mountain View, CA (US); Mariya Chavarha, Palo Alto, CA (US); Phillip Jess, San Francisco, CA (US); Marc Berndl, Mountain View, CA (US)

(21) Appl. No.: 17/925,539

(22) PCT Filed: Jun. 29, 2020
(86) PCT No.: PCT/US2020/040130

§ 371 Date: Nov. 15, 2022

(51) Int. Cl. C12N 15/10; C12P 21/02; G01N 33/543

(52) U.S. Cl. CPC C12N 15/1062; C12P 21/02; G01N 33/543

(57) ABSTRACT

This disclosure provides methods and compositions for making and using a protein or peptide array.

### TECHNICAL FIELD

This disclosure generally relates to peptide arrays and methods of making and using such peptide arrays.

### BACKGROUND

A peptide microarray consists of a collection of peptides displayed on a solid surface. They are used in functional and binding assays and serve a variety of biological and pharmaceutical uses, including enzyme profiling, antibody mapping, and biomarker discovery. Peptide arrays can be used to examine protein-protein and drug-protein interactions by screening a high number of peptides or proteins on a solid surface. However, these approaches have been expensive, low throughput relative to a human proteome, difficult and unreliable, primarily due to the limited amounts and length of peptides that are able to be synthesized directly on chips.

### SUMMARY

Methods and compositions are described herein that can be used to generate a protein or peptide array. Specifically, the methods described herein can be used to generate a multi-million to multi-billion protein/peptide array in a very short time (e.g., less than a day).

In one aspect, methods of generating an array of polypeptides are provided. Such methods generally include providing an array including a plurality of single-stranded DNAs (ssDNAs), where some or all of the ssDNAs encode a polypeptide; generating a plurality of double-stranded DNA (dsDNA) bridges, where both ends of each dsDNA are affixed to the surface of the array via one or both ssDNAs that make up each dsDNA; transcribing the plurality of dsDNA bridges to produce a corresponding plurality of RNA transcripts, where each member of the plurality of transcripts remains bound to the corresponding member of the plurality of dsDNA bridge-RNA polymerase complexes; and translating the plurality of transcripts to produce a plurality of polypeptides, where each member of the plurality of polypeptides remains bound to the corresponding member of the plurality of RNA transcript-ribosome complexes. Such methods can be used to generate an array of polypeptides.

In another aspect, methods of generating an array of polypeptides are provided. Such methods generally include providing an array including a plurality of single-stranded mRNAs (ss-mRNAs), wherein each member of the plurality of ss-mRNAs encodes for a polypeptide; and translating the plurality of ss-mRNAs to produce a plurality of polypeptides, where each member of the plurality of polypeptides remains bound to the corresponding member of the plurality of ss-mRNAs. Such methods can be used to generate an array of polypeptides.

In still another aspect, methods of generating an array of polypeptides are provided. Such methods generally include providing an array including a plurality of clonal spots of ssDNAs covalently attached to a surface of the array, where some or all of the plurality of ssDNAs encode a polypeptide; replicating the plurality of ssDNAs to generate a plurality of clonal spots of dsDNAs; transcribing the plurality of dsDNAs to produce a plurality of RNA transcripts, where each member of the plurality of RNA transcripts remains bound to a corresponding dsDNA-RNA polymerase complex; and translating the plurality of transcripts to produce a plurality of polypeptides, where each member of the plurality of polypeptides remains bound to a corresponding RNA transcript-ribosome complex. Such methods can be used to generate an array of polypeptides.

In some embodiments, the transcribing proceeds towards the surface of the array. In some embodiments, the step of providing the array includes assembling the plurality of single-stranded nucleic acid sequences on the surface of the array.

In some embodiments, the array includes known sequences at known positions. In some embodiments, the ss-mRNA is attached to the array at its 3′ end. In some embodiments, the ss-mRNA is attached to the array at its 5′ end.

In some embodiments, the array comprises a plurality of ss-mRNA-DNA-puromycin fusion molecules, where each fusion molecule includes a) an mRNA sequence containing a translation initiation sequence and an open reading frame encoding for a polypeptide attached to the solid substrate by its 5′ end, b) a DNA linker 16 to 40 nucleotides long, and c) a puromycin molecule attached to the 3′ end of the DNA linker.

In some embodiments, the plurality of polypeptides are known polypeptides, unknown polypeptides, random polypeptides, one polypeptide having a variety of mutations, computationally-generated polypeptides, or combinations thereof.

In some embodiments, transcribing comprises providing an RNA polymerase and nucleotides. In some embodiments, translating comprises providing ribosomes, tRNAs and free amino acids. In some embodiments, the transcribing and/or translating comprises providing cell lysates or standard translation mixes.

In still another aspect, methods of using a polypeptide array made by any of the methods described herein is provided. Such methods generally include contacting the array with one or more ligands; and determining whether or not the one or more ligands bind to one or more of the plurality of polypeptides on the array; and optionally, determining which one or more of the plurality of polypeptides on the array is bound by the one or more ligands.

In one aspect, methods of using a polypeptide array made by any of the methods described herein are provided. Such methods generally include contacting the array with one or more ligands, where the one or more ligands are nucleic acid-barcoded; ligating the nucleic acid barcode of the ligand to the plurality of ss-mRNA or dsDNA; determining whether or not the one or more ligands binds to one or more of the plurality of polypeptides on the array by sequencing; and optionally, determining which one or more of the plurality of polypeptides on the array is bound by the one or more ligands.

In some embodiments, the ss-mRNA or dsDNA are modified by site-specific restriction nucleases or endonucleases prior to, during, or following contacting the array with the one or more nucleic acid-barcoded ligands.

In some embodiments, the dsDNA or ss-mRNA includes a nucleic acid barcode prior to the start codon for identifying the polypeptide encoded by the dsDNA or ss-mRNA. In some embodiments, the dsDNA or ss-mRNA contains a nucleic acid barcode following the coding sequence for identifying the polypeptide.

In some embodiments, the plurality of ligands can be, without limitation, antibodies, aptamers, nucleic acids, proteins, peptides, and other small molecule binders.

In another aspect, methods of using an array of polypeptides made by any of the methods described herein are provided. Such methods generally include contacting the array with one or more substrates and reaction reagents; detecting the presence of activity by one or more of the plurality of polypeptides on the array; and optionally, determining which one or more of the plurality of polypeptides on the array exhibited activity.

In still another aspect, polypeptide arrays made by any of the methods described herein are provided.

In some embodiments, all or a portion of the DNA bridge is removed following transcription using DNA specific nuclease digestion and restriction methods. In some embodiments, all or a portion of the RNA transcript is removed following translation using RNA specific nuclease digestion and restriction methods.

In some embodiments, at least one of the plurality of polypeptides comprises a protein domain capable of ligating polypeptides to nucleic acids proximally displayed on the array. In some embodiments, at least one of the plurality of polypeptides comprises a nucleic acid-binding protein domain capable of binding nucleic acids proximally displayed on the array.

In some embodiments, at least one of the plurality of polypeptides comprises one or more cleavage sites susceptible to cleavage by site-specific proteases. In some embodiments, at least one of the plurality of polypeptides comprises a site susceptible to cleavage by at least one site-specific protease, and wherein the N-terminus of at least one of the plurality of polypeptides is modified by the addition of the corresponding site-specific proteases. In some embodiments, at least one of the plurality of polypeptides comprises a site susceptible to cleavage by a site-specific protease, wherein the plurality of polypeptides are released from the array.

In yet another aspect, polypeptides made by any of the methods described herein are provided.

In some embodiments, one or more fiducials are provided on the polypeptide array. Representative fiducials include, without limitation, one or a combination of physical markings on the array, fluorophore conjugated to a custom chip, fluorescent proteins, or fluorophore-conjugated molecules that can bind to specific components displayed on the array.

In some embodiments, the array comprising the plurality of ssDNA sequences is generated by affixing the plurality of ssDNA sequences flanked by adaptor sequences onto an array comprising a lawn of sequences wherein one set of sequences is complementary to one of the flanking adaptor sequences, and the other set of sequences is identical to the other flanking adaptors sequence.

In some embodiments, the array comprises a plurality of beads, wherein the surface of each individual bead is affixed to a plurality of copies of either a unique dsDNA sequence or multiple unique dsDNA sequences.

In some embodiments, generating the array including the plurality of dsDNA on the plurality of beads includes functionalizing the surface of the plurality of beads; attaching a plurality of oligonucleotide adapters to the functionalized surface of the plurality of beads; depositing one or more ssDNA variants on each bead; and amplifying and converting the one or more ssDNA variants into a plurality of dsDNA clones.

A peptide array as described herein can be generated in-house in one day at a fraction of the cost of purchasing a peptide array, which can take 3 to 4 weeks to obtain commercially. Significantly, peptides that are more than twenty times longer than commercially available peptides on arrays can be obtained, and using the methods described herein, long peptides or proteins can be efficiently generated on the array, thus increasing the number of peptides and proteins that can be studied on one chip by thousands fold and increasing the space of functional biological targets to investigate, such as fluorescent proteins, enzymes, nanobodies, antibodies, etc.

The methods described herein allow for controllable, accurate, and high-yield synthesis of a peptide array at a fraction of the cost. In addition to reducing labor and reagent costs, the methods described herein can be used to increase the number of unique targets on each array from about 10,000 to greater than 2.5 billion. This capability would accelerate the large-scale identification of compounds for potential use in diagnostics, therapeutics, food and environmental safety, cosmetics, protein engineering, synthetic biology, basic science research, binder discovery.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1A-1C are schematics showing one embodiment of the methods described herein.

FIG. 2 is a schematic showing one embodiment of the methods described herein in more detail.

FIG. 3 is a schematic showing one embodiment of an alternative method of generating a peptide array from a DNA microarray described herein.

FIG. 4 is a schematic showing embodiments of five variations of the methods described herein.

FIG. 5A is a fluorescent image of a flow cell after generating dsDNA and prior to incubation with a dye-conjugated oligo probe that is complementary to the RNA stall sequence universal in the library, taken as a negative control for ssDNA detection.

FIG. 5B is a fluorescent image of a flow cell detecting the presence of ssDNA after generating dsDNA and incubating the flow cell with a dye-conjugated oligo probe that is complementary to the RNA stall sequence universal in the library.

FIG. 5C-E are fluorescent images detecting the presence of the desired RNA product post-translation (FIG. 5C), expressed EGFP proteins post-transcription (FIG. 4D), and expressed FLAG peptides post-transcription (FIG. 5E).

FIG. 5F. is an overlapping image of the flow cell derived from two channels detecting EGFP and FLAG post-transcription.

FIG. 6A-D are fluorescent images of wherein the flow cells were incubated with RNAP loading (FIGS. 6A and 6B), or directly with the full Transcription Mix (FIGS. 6C and 6D). Flow cells were probed and imaged to detect 3×FLAG RNA probed with a complementary oligo conjugated with dye (FIGS. 6A and 6C) and peptide expression probed with fluorescent antibody (FIGS. 6B and 6D).

FIG. 6E is a chart comparing the mean fluorescence intensity of RNA probe or protein-detecting antibody probes on chips with and without RNAP loading.

FIG. 7A-B are fluorescent images detecting the presence of the desired RNA product post-translation (FIG. 7A) and EmGFP fluorescence alone (FIG. 7B).

FIG. 7C is an overlapping image of the flow cell derived from two channels detecting EmGFP RNA probe and EmGFP.

### DETAILED DESCRIPTION

A protein or peptide microarray, also known as a peptide array, is a collection of proteins or peptides displayed on a surface (e.g., glass, silicon, gel, or plastic surface), and can be used in binding assays and functional assays. Protein or peptide arrays serve a variety of biological and pharmaceutical uses including being used in enzyme profiling, bioengineering, antibody mapping, and biomarker discovery. Current methods that are used for performing such binding specificity experiments, however, each have at least one significant limitation related to cost, time, difficulty, flexibility and/or throughput, particularly when trying to evaluate many measurement points of a billion or more different unique protein variants. The human genome contains 20,000-25,000 genes which could code for greater than 100,000 different transcripts and produce an estimated 1 million different protein variants. Processes such as post-translational modifications, alternative splicing, colocalization, protein complex formation, and degradation regulate protein activities over time and require systems level interrogation of the proteome to understand the biological state. The methods described herein have been developed so as to be able to construct a multi-million- to billion-spot protein or peptide array in a day or less to enable high-throughput assays.

Protein arrays or peptide arrays can be made using the compositions and methods provided herein. As used herein, “protein” refers to one or more covalently bound polypeptides with a total of 50 or more amino acids; often referring to full-length polypeptides whereas “peptide” refers to a short chain of 2 to 50 amino acids, which can be a fragment of a full-length protein or an enzymatically functional polypeptide. Shorter amino acid chains of peptides result in less well-defined features, whereas proteins that have longer amino chains that can form secondary, tertiary, and quaternary structures that can change conformation in response to its environment and binding events. As used herein, “array” refers to a collection of clonal clusters of molecules arranged in an orderly fashion attached to a solid or semi-solid surface including, but not limited to, glass, silicon, gel (e.g. agarose, polyacrylamide, etc.), or plastic surfaces.

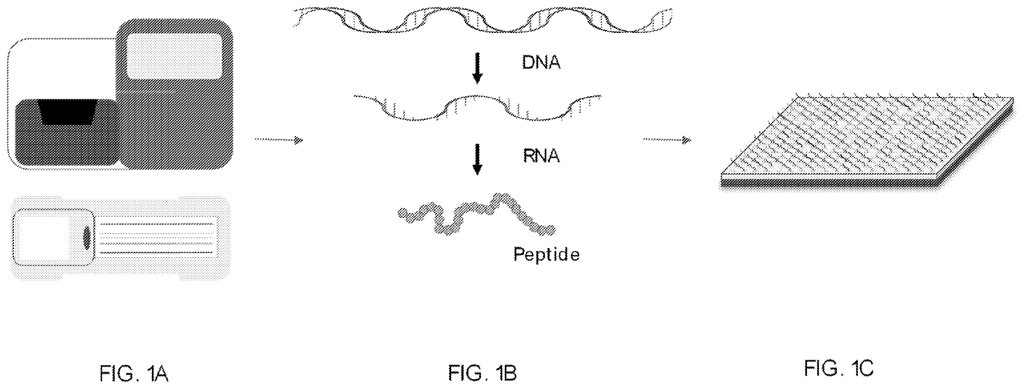

To generate an array of proteins or peptides, a library of single-stranded DNA (ssDNA) is deposited or attached and sequenced on a flow cell using conventional NGS technology (FIG. 1A). There are several ways to initially generate a DNA array including direct synthesis on glass, PCR amplification of hybridized DNA molecules to an adaptor, or chemical attachment of a library of DNA oligos on a modified surface. Once a DNA library is present on the surface of the glass, the DNA oligos can either be converted to RNA and proteins directly or else amplified into a homogenous DNA spot to improve signal to noise of downstream binding events to the subsequently transcribed RNA and translated peptides/proteins. If the binding event will be visualized, there are two approaches for imaging: (a) single molecule imaging, where a single DNA oligo would be translated into an RNA transcript and then translated into a single protein or peptide (thus presenting with a single binding event between a target and binder), or (b) cluster imaging of a spot greater than the diffraction limit, where a single DNA oligo is amplified into a cluster of homogenous DNA oligos, transcribed into RNA transcripts and translated into a peptide or protein cluster (where multiple binding events of the sample peptide or protein molecule would occur). Amplification of the initial attached oligonucleotide DNA library using PCR allows high-throughput arrays to be built quicker, cheaper, and faster compared to traditional methods of synthesizing proteins for binding assays. In our assay, we utilize a DNA Next Generation Sequencing (NGS) instrument (e.g. Illumina MiSeq) to create DNA clusters by hybridizing the original DNA library, and to expand the single DNA oligos into a cluster. The steps of annealing a DNA library to a solid surface and expansion of the single DNA oligos into a cluster could also be generated with a flow cell and PCR instrument.

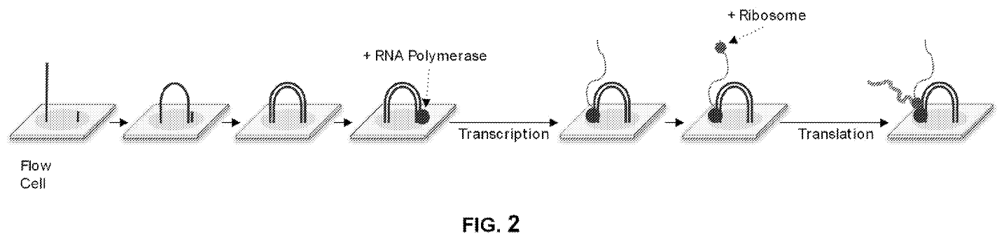

One critical step in our protein/peptide array generation assay is orientation of the DNA constructs during cluster generation. Our modified DNA cluster generation protocol requires reprogramming the Illumina sequencing instrument to halt cluster generation during the paired-end turnaround step after the second set of bridge amplification, preserving the dsDNA bridge that is already generated during the bridge amplification step (FIG. 2C). Following the sequencing of the nucleic acid (revealing the precise sequence information for each geographic location on the DNA array) and formation of the bridge, transcription and translation reagents are provided to produce proteins from the originally sequenced and assembled DNA constructs (FIG. 1B). As the DNA constructs lack an RNA polymerase terminator sequence and also contain a ribosome stalling motif, the protein, RNA, ribosome and RNAP all remain associated with each other and the DNA bridge, thereby forming a peptide array (FIG. 1C). Alternatively, in addition to ribosome stalling motif, the DNA construct may also lack a stop codon to limit dissociation of the ribosome and translated peptide from the RNA transcript.

FIG. 2 is a schematic showing the process of generating a single peptide or protein from a bridged nucleic acid covalently bound to a solid substrate. The addition of RNA polymerase, as well as any other necessary transcription reagents, results in the production of an RNA molecule that remains attached to the transcription complex, at least in part because the RNA polymerase is halted and blocked by the solid substrate at the other end of the nucleic acid. Similarly, the addition of the reagents necessary for translation results in the production of a protein or peptide molecule that remains attached to the RNA via the ribosome. In this way, a peptide array is produced, containing clusters of peptides/proteins of known sequences. Specific positions of each peptide or protein cluster on the array is provided by the sequencing data of their associated DNA construct.

In the methods described herein, the localized amplification reaction which generates clonal clusters of DNA is halted after generating clusters of dsDNA bridges and prior to the step that linearizes and denatures the DNA strands, thereby obviating a separate manual second strand synthesis step (FIG. 2). A bridge is formed when a single-stranded oligonucleotide folds over and hybridizes to an adjacent, immobilized complementary oligonucleotide, hereafter referred to as a lawn primer. It becomes double stranded when the complementary strand is generated by extending the 3′ ends of the lawn primers. A bridge is shown schematically by an upside down “u”-shaped nucleic acid tethered on both sides to a flow cell (FIG. 2). This important feature overcomes key technical limitations of the “Prot-MaP” methods²:
Biotin/streptavidin binding system is inefficient due to competition with adapter on substrate that can decrease dsDNA yield
Streptavidin can cause crosslinking, which may reduce yield due to steric hindrance
Biotin/streptavidin binding system is bulky, which may hinder ligand binding
Synthesis of second DNA strand off the sequencer requires more manual labor and time, and experimental variance
Synthesis of second DNA strand off the sequencer can be inefficient for longer DNA oligonucleotides
Variable protein yield due to complicated manual protocols for RNA transcription

As described herein, oligonucleotides of known sequences can be hybridized onto the flow cell such that their positions can be ascertained to serve as reference points. These reference points can be used to align images of the same flow cell at different times to register the location of specific products at different stages, for example, prior to or after DNA transcription, or after peptide translation. Probed peptides of interest can be matched to the location, and therefore the identity, of its progenitor DNA. Alternatively, nucleic acids of known sequence may be provided in the initial library preparation, such that once deposited and amplified on the chip, transcribed and/or translated, their location can be determined using fluorescent oligonucleotide probes directed against the specific RNA transcripts or using binding reagents to the translated polypeptides. Similarly to the reference oligonucleotides these then would serve as reference points to register the location of protein clusters on the array.

Transcription of DNA into RNA is well known in the art, and the mechanisms behind various RNA polymerase enzymes also are well known in the art. Translation of RNA into proteins also is well known in the art, and cell free systems have been described that provide all the necessary components for transcription and translation to take place outside of a whole cell. Similarly, the components necessary for transcription and translation can be provided (e.g., flowed onto the chip) such that the reactions necessary for transcription and translation take place on the bridged nucleic acid as described herein.

Building a bridged dsDNA system removes the need for any type of binding system (e.g., streptavidin/biotin) needed to stall RNA polymerase on the DNA strand, and reduces the number of manual steps by over half, resulting in greater yield of functional clusters, robustness in sample size, consistency, and convenience. A double-stranded DNA bridge system to generate DNA clusters avoids any loss of dsDNA generation efficiency arising from DNA crosslinked via any binding system used to stop the RNA polymerase. Lastly, the methods described avoid incorporating toxic chemical reagents since this protocol bypasses steps with sodium hydroxide or formamide to remove residual primers or read fragments. Therefore, the methods described herein are much more efficient while producing more consistent results than current methods.

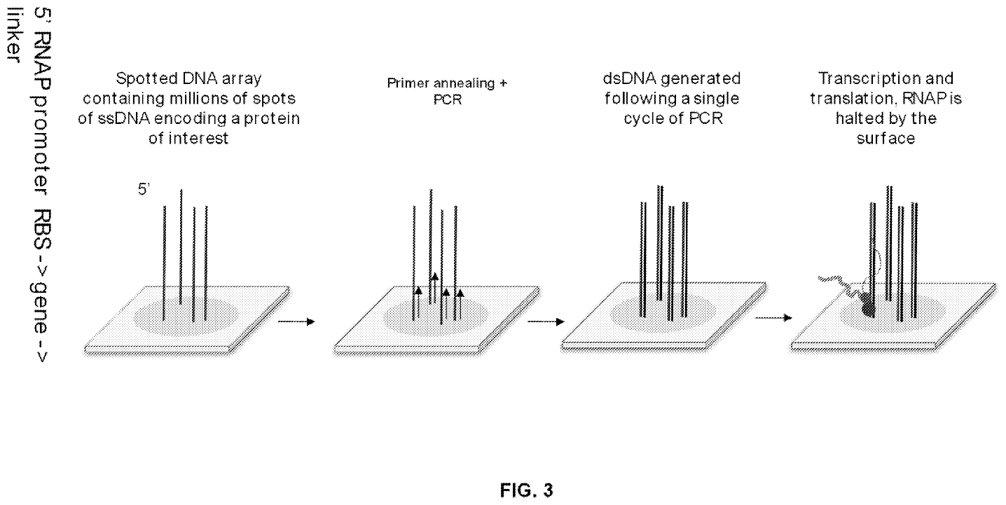

It is important to note that in the disclosed invention the RNA polymerase is halted and blocked by the solid substrate at the other end of the bridged dsDNA. This provides for a convenient and easy way to immobilize DNA and RNA, and subsequently the produced protein and RNA all together, simply by ensuring the directionality of the RNA polymerase promoter. This approach can be easily extended to a linear dsDNA (for example purchased as commercially available microarrays) by ensuring that transcription proceeds from the top of the DNA molecule down towards the slide as depicted in FIG. 3. While a number of different technologies are known for generating proteins displayed on 2D surfaces that utilize cell-free protein expression, they all rely on capturing produced proteins via an affinity tag (e.g. His or GST tags) to affinity reagents pre-spotted onto the array².

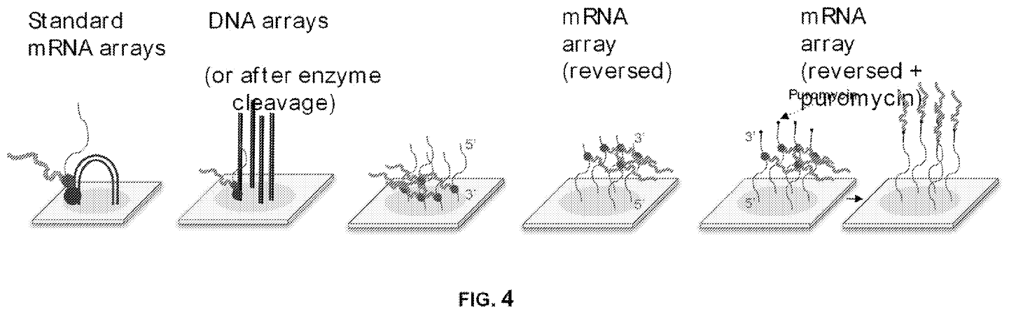

An alternative method of producing a peptide array eschews the DNA to RNA transcription process by directly attaching an mRNA construct consisting of at least a translation initiation sequence and a sequence encoding for the desired polypeptide or protein onto a solid substrate. In another embodiment, clonal spots of an mRNA construct sequence, rather than single molecules, can be derived through spotted mRNA arrays. Cell-free translation of the mRNA construct would be performed as described above (FIG. 4). Additionally, a peptide array of DNA barcoded polypeptides and proteins can be generated if the construct displayed on the solid substrate consists of 3 regions covalently linked in the following order from 5′ to 3′:
1. mRNA consisting of at least a ribosome binding site, translation initiation sequence, and an open reading frame encoding for a polypeptide or protein,
2. DNA linker which may contain a unique barcode, attached to the 3′ end of the mRNA, and which is flexible and long enough for the puromycin to enter the ribosome A site, and
3. puromycin molecule attached to the 3′ end of the DNA linker.
   As the ribosome translates the mRNA into a polypeptide, it stalls at the junction between RNA and DNA. The puromycin enters the A site of the ribosome, forming a nascent peptide chain covalently attached to the puromycin and causing the release of the ribosome³.

The methods described herein are significantly improved over existing methods at least because:
Protein or peptide targets greater than 10-20 amino acids can be produced using the methods described herein. Most conventional peptide array techniques are limited to on-glass peptide synthesis of 10-20 amino acids, however, the methods described herein have been demonstrated to produce green fluorescent protein (EmGFP), which is 239 amino acids in length. Additionally, longer proteins have complex secondary structure, and the methods described herein also have been successfully demonstrated to not only generate longer targets, but also functional targets with secondary structure (e.g., a functional EmGFP molecule).
The methods of making peptide arrays described herein can use an automated sequencer (e.g., MiSeq, HiSeq) and don't require a separate manual second DNA strand synthesis. The methods described herein allow for the development of surface protein or peptide binding assays that allow for visual identification of bound molecules based on the location on the chip. Unlike Prot-MaP, the methods described herein can generate the second DNA strand in the same step as assembling and sequencing of the DNA, cutting the number of manual steps by more than half while providing a higher yield of dsDNA.
The peptide arrays described herein allow high-throughput assays unlike any current peptide array. The peptide arrays described herein each can contain, and, therefore, be used to screen, millions to billions of protein variants, rather than the thousands that current commercial peptide arrays contain.
The peptide arrays described herein can be used with multiplex targets in multiplex experiments (e.g., DNA- or RNA-barcoded protein or peptide targets). The peptide arrays described herein can be used with multiple compounds in multi-multi experiments (e.g., ligate barcoded DNA-tagged diverse small molecule to RNA; peptide and PCR for linked DNA:RNA sequence; or compounds with different dye molecules and microscopy (see, for example, Moffitt et al., 2016, Methods Enzymol., 572:1-49⁴)).
The cluster density on the peptide arrays described herein can be varied by modifying the library loading concentration as per NGS library preparation guidelines, while cluster size and/or density itself can be varied by modifying the number of cycles during the sequencing run, or varying the concentration of enzymes or reagents.
The peptide arrays described herein allow for quality control and specificity assays (e.g., obtaining aptamer binding data in between each round of SELEX for machine learning analysis).

The peptide arrays described herein can be used to examine binding between any number of targets (e.g., biological targets or non-biological targets). For example, the peptide arrays described herein can be used to examine interactions with any number of compounds (sometimes referred to as binders) including, without limitation, antibodies, antigens, aptamers, cell surface markers, DNA molecules, proteins, peptides, RNA molecules, and/or small molecules.

The source of the proteins or peptides on the arrays described herein, or the source of the nucleic acids encoding the proteins or peptides on the arrays described herein, can be obtained from virtually any source. For example, nucleic acids encoding major histocompatibility complex (MHC) proteins can be used to populate an array as described herein, or nucleic acids contained within a biome can be used to populate an array as described herein.

Generally, at least one of the molecules (one or more proteins or peptides on the array and/or the compound(s) to which the array is being exposed) includes a label that can be detected visually or otherwise such as: (a) fluorescent dye, (b) one or more hybridizable or ligatable oligonucleotides, (c) acceptor(s) and corresponding donor(s) for Förster Resonance Energy Transfer (FRET), or (d) radioactivity. For example, the methods described herein can be used to screen for RNA, or synthetic nucleic acids such as LNA or TNA, aptamers, small molecule targets or protein complexes that bind to peptides. In some instances, a barcoded segment can be attached to one or more proteins or peptides on the array and/or the compound(s) to which the array is being exposed.

While embodiments for generating peptides and proteins on the array described herein utilized MiSeq next generation sequencing platform, the plurality of dsDNA sequences may also be generated on custom surfaces, including but not limited to:
spotted DNA microarrays. For example, DNA arrays are commercially available or generated in-house with a DNA spotter, including arrays wherein the physical location of each DNA sequence is known on the array. Longer ssDNA sequences may be assembled by directed ligation on the chip from shorter pieces. In the case of commercially-obtained arrays, a library of clusters containing identical ssDNA wherein the 3′ end is attached to the glass is provided, and dsDNA generation in a bridge conformation is not possible due to a lack of a lawn of oligo adapters. Instead, the dsDNA template for transcription can be easily generated by a single cycle of primer annealing and 3′ DNA extension using DNA polymerase (or fragments) lacking 5′ to 3′ exonuclease activity. Following the dsDNA generation, RNAP is initiated on the dsDNA template and transcribes RNA until halted by the substrate surface. Translation would proceed identically to the embodiment in which the DNA is in the bridge conformation (FIG. 3).
custom chips with a lawn of oligo primers. Library of ssDNA molecules may be affixed to the array surface by utilizing adapter sequences on either end of the ssDNA molecule that have the same or complementary sequence to a lawn of oligos on the custom chip. Once deposited onto the chip, ssDNA molecules can be converted to dsDNA in a bridge conformation using PCR, and their sequences can be determined using custom optical microscopy setup.
beads. Instead of an array, the dsDNA molecules can also be generated on beads such that a single DNA variant is deposited per bead. By functionalizing bead surfaces with a lawn of oligonucleotide adapters, similarly to the array, ssDNA can be amplified and converted to dsDNA. These dsDNA molecules can be transcribed and translated, and the produced polypeptides or proteins can be interrogated by methods like FACS which would permit isolation of the desirable polypeptide or protein variants.

Additionally, a number of modifications can be made to the methods described herein to adapt the methods to different applications. For example, for protein engineering optimization, a degenerate, random or machine learning (ML)-modeled library can be used to generate the initial array, which then allows for testing many different proteins very quickly. To detach the peptide from the bulky ribosome-RNA polymerase-DNA complex, it is possible to link peptides to unique DNA adapters fixed on a flow cell, for example via puromycin or endonucleases, and dissociate the peptide from the ribosomal complex. This linkage would maintain presentation of the peptide to a solid substrate. Such a configuration would generate fixed peptides with short DNA barcodes. Additionally or alternatively, a step can be added to remove the RNA and/or DNA to decrease non-specific binding of targets to the nucleic acids. It is also possible to re-use the initial dsDNA array by incubating the generated peptide array with ribonucleases and proteases to degrade the RNA, RNA polymerase, ribosome, peptide/protein, and any therefore any ligands binding to these elements.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1—EGFP and FLAG Peptide Array

### Materials

DNA sequencing, cell-free transcription and translation was performed on NextSeq or MiSeq Reagent Kits, supplemented with PhiX Control v3, and sequenced on a MiSeq500 (Illumina). A custom PURExpress Kit In Vitro Protein synthesis kit which lacked CTP and UTP in Solution A, and T7 RNAP and RF1, RF2, and RF3 in Solution B was commercially obtained from New England BioLabs. Post-transcription and -translation washes were performed with PBST+MgCl₂ buffer (1×PBS, 7 mM MgCl₂, and 0.05% Tween-20 in nuclease free water). Dye-conjugated oligos used to detect RNA products were purchased from IDT. Proteins were probed with GFP Tag Polyclonal Antibody Alexa Fluor 488 and DYKDDDDK Tag Monoclonal Antibody (L5), Alexa Fluor 555 purchased from Thermo Fisher Scientific. All fluorescence imaging was performed on a custom-built ASI microscope.

### Libraries

EGFP gBlocks® Gene Fragments and 2×FLAG gBlocks® Gene Fragments were purchased from IDT. The constructs are designed to contain the following components: a) P5 adaptors, b) RNA polymerase (RNAP) promoter, c) RNAP stall site (38 bp), d) Shine Dalgarno sequence, e) start codon, f) Read 1 sequencing primer hybridization site (2×FLAG only), g) protein coding region, h) linker with no stop codon (18 bp), i) Read 2 sequencing primer hybridization site, j) coding region for peptide spacer sequence (99 bp), k) ribosome stall sequence (81 bp), l) unstructured RNA, and m) roadblock sequence and P7 adaptor.

### Methods

Library Sequence Preparation and dsDNA Synthesis

4 μL of 4 nM EGFP gBlocks® Gene Fragments and 1 μL of 4 nM 2×FLAG gBlocks® Gene Fragments were combined and denatured. 5 μL of the prepared library was incubated with freshly prepared 5 μL of 0.2N NaOH in a microcentrifuge tube for 5 minutes at room temperature. The solution was mixed with 990 μL of prechilled HT1 buffer. 630 μL of this solution was then mixed with 70 μL of 20 pM PhiX to make the pre-sequencing mix. 680 μL of the pre-sequencing mix was added to the sample port. 3.4 μL of the sequencing primers, EGFP (100 μM) and FLAG rd1 (100 μM) were added to the Port 12, for a final concentration of 0.5 μM. The flow cell was washed with nuclease free water, ethanol, and wiped with Kimwipes.

The libraries were sequenced on an Illumina MiSeq500 with a modified protocol to halt the run during the paired-end turnaround step. After the sequencing run was complete, the flow cell was stored in 4° C. in lx PBS until used.

Assessing dsDNA Generation Efficiency

Low presence of ssDNA was verified with Fluorescence In Situ Hybridization assay (FISH) by incubating the flow cell with a Cy3-conjugated oligo probe that is complementary to the RNA stall sequences in the DNA library after halting the MiSeq run during the paired-end turnaround step, which should produce no signal if the ssDNA was effectively converted to dsDNA. If this step is done any time after RNA transcription, the RNA must be degraded by RNase before the FISH assay is performed.

### Transcription

The flow cell was incubated with 300 μL of 1×E. coli polymerase buffer for 5 to 15 minutes at room temperature. 100 μL of 1o Transcription Mix lacking CTP nucleotide (1×E. coli polymerase buffer, 0.02 mg/mL BSA, 1.5% glycerol, 25 μM ATP, 25 μM GTP, 25 μM UTP, and 125 unit/mL RNA polymerase in nuclease free water) was flowed into the flow cell, and incubated for 30 minutes at 37° C. while wrapped in parafilm. After incubation with 1o Transcription Mix, the flow cell was washed with 400 μL of Transcription Wash Mix (1×E. coli polymerase buffer, 0.02 mg/mL BSA, 1.5% glycerol, 25 μM ATP, 25 μM GTP, and 25 μM UTP in nuclease free water). Then 200 μL of 2o Transcription mix (1×E. coli polymerase buffer, 0.02 mg/mL BSA, 1.5% glycerol, 1 mM ATP, 1 mM GTP, 1 mM UTP, and 1 mM CTP in nuclease free water) was added to the flow cell and incubated for 1 hour at 37° C. while wrapped in parafilm. The flow cell was washed with 500 μL PBST+MgCl₂ buffer twice.

### Translation

A custom PURExpress Kit reaction mixture that lacked CTP and UTP in Solution A, and T7 RNAP and RF123 in Solution B was assembled on ice according to manufacturer's instructions to a final volume of 100 μL (40 μL Solution A, 30 μL Solution B, 4 μL Superase inhibitor, and 26 μL nuclease free water) and added to the flow cell for a 1 hour incubation at 37° C. while it was wrapped in parafilm. Then the flow cell was washed with 500 μL PBST+MgCl₂ buffer twice.

### Detecting RNA

Presence and quantity of RNA was verified with FISH by incubation with RNAP_stall_647, a dye-conjugated oligo that is complementary to the RNAP stall sequence on the RNA, and imaging after dsDNA transcription to RNA.

### Detecting Protein

The flow cell was incubated with antibody staining buffer (1×PBS, 7 mM MgCl₂, 0.05% Tween-20, and 10 mg/mL BSA in nuclease free water) for 10 minutes at room temperature to pre-block flow cell components, then with 10 μg/mL Anti-EGFP 488 and Anti-FLAG 555 primary antibody in staining buffer for 30 minutes at room temperature. The stained flow cell was washed twice with 500 μL PBST+MgCl₂ buffer at room temperature and imaged on a custom ASI widefield fluorescence microscope.

### Results

Assessing dsDNA Generation Efficiency

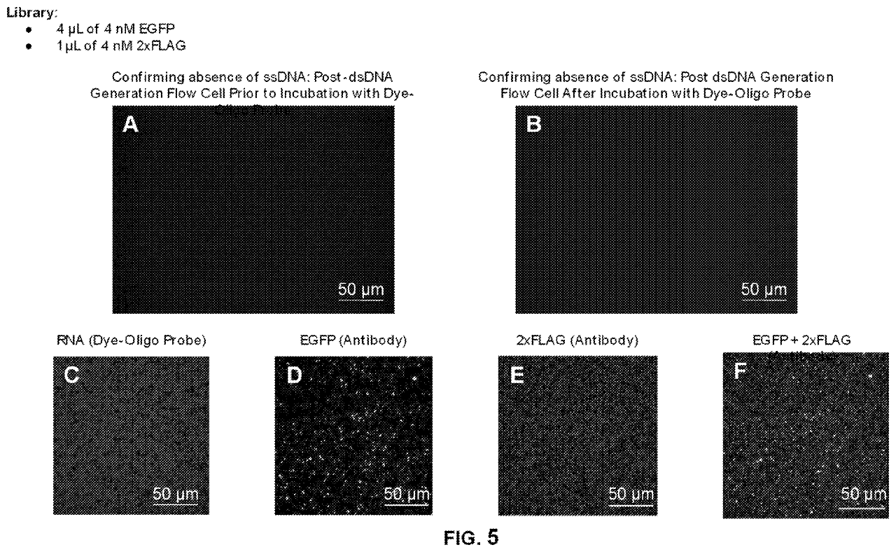

FISH assay images of flow cell incubated Cy3-conjugated oligo probe that is complementary to the RNAP stall sequences in the DNA library after halting the MiSeq run during the paired-end turnaround step shows low fluorescent signal, indicating high dsDNA generation efficiency (FIGS. 5A and 5B).

### Transcription Efficiency

Fluorescent imaging of the flow cell incubated with dye-conjugated oligo complementary to the RNAP stall sequence on RNA post-translation revealed presence of RNA, thereby confirming efficient dsDNA generation and transcription (FIG. 5C).

### Translation Efficiency

Immunofluorescence assay shows moderate EGFP (FIG. 5D) and high 2×FLAG (FIG. 5E) expression. An image overlapping the fluorescent signal from both antibodies indicates much higher 2×FLAG expression compared to EGFP (FIG. 5F).

### Example 2—Peptide Array with 3×FLAG Showing Improved Transcription and Translation Efficiency

### Materials

DNA sequencing, cell-free transcription and translation was performed on NextSeq or MiSeq Reagent Kits, supplemented with PhiX Control v3, and sequenced on a MiSeq500 (Illumina). A custom PURExpress Kit In Vitro Protein synthesis kit which lacked CTP and UTP in Solution A, and T7 RNAP and RF123 in Solution B was commercially obtained from New England BioLabs. Post-transcription and -translation washes were performed with PBST+MgCl₂ buffer (1×PBS, 7 mM MgCl₂, and 0.05% Tween-20 in nuclease free water). Dye-conjugated oligos used to detect RNA products were purchased from IDT. Proteins were probed with Monoclonal ANTI-FLAG® M2 antibody purchased from Sigma and Goat anti-Mouse IgG (H+L) Highly Cross-Adsorbed Secondary Antibody, Alexa Fluor Plus 555 purchased from Thermo Fisher Scientific. All fluorescence imaging was performed on a custom Nikon widefield fluorescence microscope.

### Libraries

3×FLAG gBlocks® and EmGFP Gene Fragments were purchased from IDT. The 3×Flag gBlocks® Gene Fragment construct contains a) P5 adaptors, b) RNA polymerase (RNAP) promoter, c) RNAP stall site (38 bp), d) Shine Dalgarno sequence, e) start codon, f) Read 1 sequencing primer hybridization site, g) protein coding region, h) linker with no stop codon (18 bp), i) Read 2 sequencing primer hybridization site, j) coding region for peptide spacer sequence (99 bp), k) ribosome stall sequence (81 bp), l) unstructured RNA, and m) roadblock sequence and P7 adaptor. The 3×FLAG peptide sequence was DYKDHDGDYKDHDIDYKDDDDK.

The EmGFP Gene Fragment construct used for the validation of peptide array synthesis was designed to contain the following components: a) P5 adaptors, b) RNA polymerase (RNAP) promoter, c) RNAP stall site (38 bp), d) Shine Dalgarno sequence, e) start codon, f) protein coding region encoding N-terminal genetic fusion of superFLAG peptide (sFLAG) to Emerald Green Fluorescent Protein (EmGFP), g) linker with no stop codon (18 bp), h) Read 2 sequencing primer hybridization site, i) coding region for peptide spacer sequence (99 bp), j) ribosome stall sequence (81 bp), k) unstructured RNA, and l) roadblock sequence and P7 adaptor.

### Methods

Library Sequence Preparation and dsDNA Synthesis of 3×FLAG

Two chips were prepared to compare transcription and translation efficiencies. To prepare the library for both chips, 1 μL of 0.03 nM 3×FLAG gBlocks® Gene Fragments was combined with 4 ul of nuclease-free water. 5 μL of the prepared libraries were incubated with freshly prepared 5 μL of 0.2N NaOH in a microcentrifuge tube for 5 minutes at room temperature. The solutions were mixed with 990 μL of prechilled HT1 buffer. 630 μL of the solutions were then mixed with 70 μL of 20 pM PhiX to make the pre-sequencing mixes. 680 μL of the pre-sequencing mixes were added to the sample ports. 3.4 μL of the sequencing primers FLAG rd1 (100 μM) was added to the Port 12, for a final concentration of 0.5 μM. The flow cells were washed with nuclease free water, ethanol, and wiped with Kimwipes.

The libraries were sequenced on an Illumina MiSeq500 with a modified protocol to pause the run during the paired-end turnaround step. After the sequencing run was complete, the flow cell was stored in 4° C. in 1×PBS until used.

### Transcription of 3×FLAG

Both flow cells were incubated with 300 μL of 1×E. coli polymerase buffer for 5 to 15 minutes at room temperature.

For Chip 1 a two-step transcription incubation was performed. 100 μL of 1o Transcription Mix lacking CTP nucleotide (1×E. Coli polymerase buffer, 0.02 mg/mL BSA, 1.5% glycerol, 25 μM ATP, 25 μM GTP, 25 μM UTP, and 125 unit/mL RNA polymerase in nuclease free water) was flowed into the flow cell and incubated for 3 hours minutes at 37° C. while wrapped in parafilm, hereafter referred to as RNAP loading. After incubation with 1o Transcription Mix, the flow cell was washed with 400 μL of Transcription Wash Mix (1×E. coli polymerase buffer, 0.02 mg/mL BSA, 1.5% glycerol, 25 μM ATP, 25 μM GTP, and 25 μM UTP in nuclease free water). Then 200 μL of 2o Transcription Mix (1×E. Coli polymerase buffer, 0.02 mg/mL BSA, 1.5% glycerol, 1 mM ATP, 1 mM GTP, 1 mM UTP, and 1 mM CTP in nuclease free water) was added to the flow cell and incubated for 1 hour at 37° C. while wrapped in parafilm. For Chip 2, instead of the two-step incubation, 100 ul of the full Transcription Mix (1×E. Coli polymerase buffer, 0.02 mg/mL BSA, 1.5% glycerol, 1 mM ATP, 1 mM GTP, 1 mM UTP, and 1 mM CTP in nuclease free water) was flowed into the flow cell, and incubated for 3 hours at 37° C. while wrapped in parafilm.

Prior to translation, presence of RNA was detected with FISH by incubation with a dye-conjugated oligo probes after dsDNA translation to RNA (RNAP stall 647, complementary to the RNAP stall sequence on the RNA sequences for 3×FLAG). Both flow cells were washed with 500 μL PBST+MgCl₂ buffer twice.

### Translation of 3×FLAG

A custom PURExpress Kit reaction mixture that lacked CTP and UTP in Solution A, and T7 RNAP and RF123 in Solution B was assembled on ice according to manufacturer's instructions to a final volume of 100 μL (40 μL Solution A, 30 μL Solution B, 4 μL Superase inhibitor, and 26 μL nuclease free water) and added to the flow cell for a 3 hour incubation at 37° C. while it was wrapped in parafilm. Then the flow cell was washed with 500 μL PBST+MgCl₂ buffer twice.

### Detecting 3×FLAG Peptide

The flow cells were incubated with antibody staining buffer (1×PBS, 7 mM MgCl₂, 0.05% Tween-20, and 10 mg/mL BSA in nuclease free water) for 10 minutes at room temperature to pre-block flow cell components, then incubated for 60 min with 10 μg/mL Monoclonal ANTI-FLAG® M2 antibody in antibody in staining buffer at room temperature. Following incubation with the primary antibody, flow cells were washed twice with 500 μL PBST+MgCl₂ buffer and incubated with 10 μg/mL (in antibody staining buffer) of Goat anti-Mouse IgG (H+L) Highly Cross-Adsorbed Secondary Antibody, Alexa Fluor Plus 555 for 30 minutes at room temperature. The flow cells were washed with 500 μL PBST+MgCl₂ buffer twice and imaged on a custom Nikon widefield fluorescence microscope.

### Analysis of 3×FLAG Transcription and Translation Efficiency

Quantification of the transcription and translation efficiency was performed by analyzing the intensity of the labels associated with each cluster. Images were imported into a software package where each cluster in the image was identified and their positions recorded. In each fluorescent channel used, representing the different labels/processes, the center and diameter of the cluster was identified and the image pixels within an area around the centroid, based on the diameter, were summed. The mean local background around each cluster was measured by summing a set of pixels just beyond the diameter of the cluster and subsequently dividing by the total number of pixels used to measure the background. The mean local background is multiplied by the number of pixels used to sum the cluster intensity and subtracted from the summed intensity of the cluster to produce the background corrected intensity. This was performed for every cluster so a mean measure of transcription and translation efficiency can be produced by taking the mean intensity of the clusters in the appropriate fluorescent channels.

### Verification of Functional GFP Synthesis

To verify the peptide array synthesis method can produce properly folded, full length functional proteins, GFP synthesis was performed according to the methods of Chip 2 wherein, post-dsDNA synthesis, the chip was incubated directly with the full transcription mix for 3 hours. To prepare the library, 1 μL of 0.05 nM EmGFP gBlocks® Gene Fragment was combined with 4 ul of nuclease-free water. Library preparation, sequencing, on-chip transcription and translation were performed according to the methods of Chip 2. Following translation, the flow cell was washed two times with 500 μL PBST+MgCl₂ buffer and directly imaged to detect intrinsic EmGFP fluorescence.

### Results

### 3×FLAG Transcription and Translation Efficiency

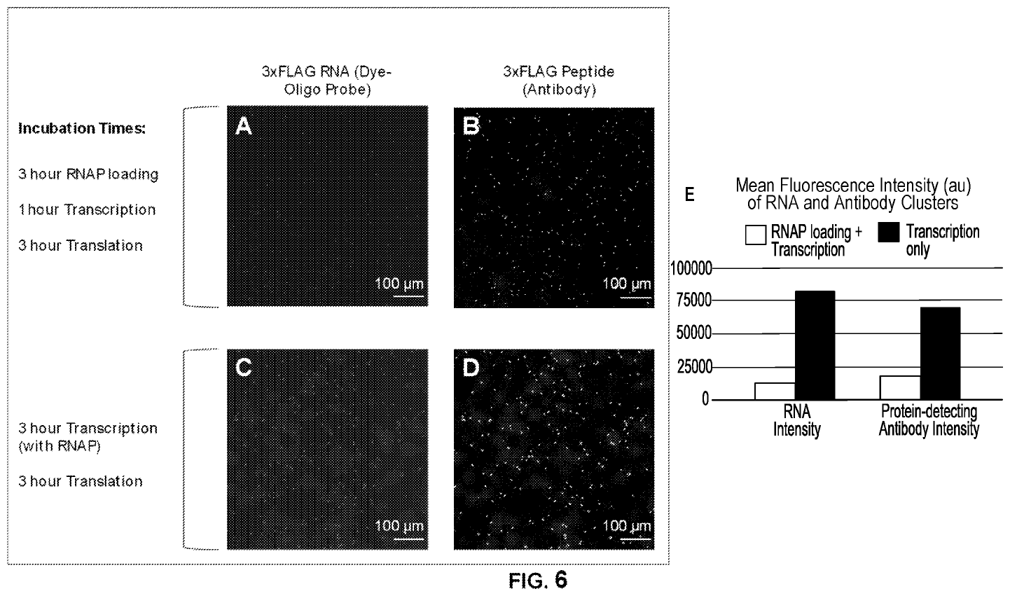

Post-transcriptional fluorescence imaging of flow cells incubated with dye-conjugated oligo complementary to the RNAP stall sequence on RNA revealed the presence of RNA (FIGS. 6A and 6C). As DNA encoding the EmGFP construct was loaded onto the chip at a lower concentration compared to Example 1, the signal from RNA and protein appears in distinct, sparse clusters. RNA labeling with oligo-dye complement shows improved transcription efficiency when the flow cell is incubated with the full Transcription Mix for 3 hours (FIGS. 6A and 6C). The result of the immunofluorescence assay detecting 3×FLAG peptide also shows that directly incubating the flow cell with the full Transcription Mix for 3 hours (FIG. 6D), instead of conducting RNAP loading (FIG. 6B), substantially increases RNA signal by over 6 fold and peptide signal by over 3 fold (FIG. 6E).

### Transcription and Translation Efficiency of Functional GFP

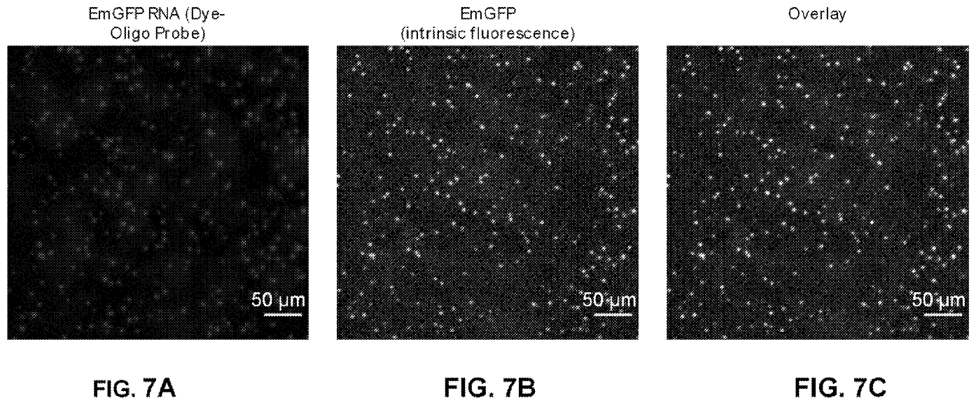

Fluorescence imaging of flow cells incubated with ATTO 647 dye-conjugated oligo complementary to the RNAP stall sequence on the RNA product revealed efficient transcription. Direct imaging of EmGFP excited at 488 shows visible clusters of autofluorescent EmGFP proteins, demonstrating that the methods herein is capable of producing full length functional proteins attached to a solid substrate (FIG. 7B). Overlaying the images from the two channels exhibits colocalization of the RNA and protein clusters (FIG. 7C). This strongly suggests that in the 488 channel signal comes from the protein produced from cell free conditions rather than from extraneous non-specific material, and that assays conducted on the array produced by the methods herein can match the location of signals from the microscope to the location of the sequenced DNA constructs.

### REFERENCES

1. Layton, C. J., Mcmahon, P. L., & Greenleaf, W. J. (2019). Large-Scale, Quantitative Protein Assays on a High-Throughput DNA Sequencing Chip. Molecular Cell, 73(5). doi: 10.1016/j.molcel.2019.02.019
2. Contreras-Llano, L. E., & Tan, C. (2018). High-throughput screening of biomolecules using cell-free gene expression systems. Synthetic Biology, 3(1). doi: 10.1093/synbio/ysy012
3. Wang, R., Cotten, S. W., & Liu, R. (2011). mRNA Display Using Covalent Coupling of mRNA to Translated Proteins. Ribosome Display and Related Technologies Methods in Molecular Biology, 87-100. doi: 10.1007/978-1-61779-379-0_6
4. Moffitt, J., & Zhuang, X. (2016). RNA Imaging with Multiplexed Error-Robust Fluorescence In Situ Hybridization (MERFISH). Visualizing RNA Dynamics in the Cell Methods in Enzymology, 1-49. doi: 10.1016/bs.mie.2016.03.020

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are the products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

1. A method of generating an array of polypeptides, comprising:
(a) providing an array comprising a plurality of single-stranded DNAs (ssDNAs), wherein some or all of the ssDNAs encode a polypeptide;
(b) generating a plurality of double-stranded DNA (dsDNA) bridges, wherein both ends of each dsDNA are affixed to the surface of the array via one or both ssDNAs that make up each dsDNA;
(c) transcribing the plurality of dsDNA bridges to produce a corresponding plurality of RNA transcripts, wherein each member of the plurality of transcripts remains bound to the corresponding member of the plurality of dsDNA bridge; and
(d) translating the plurality of transcripts to produce a plurality of polypeptides, wherein each member of the plurality of polypeptides remains bound to the corresponding member of the plurality of RNA transcripts,
thereby generating an array of polypeptides.

2. A method of generating an array of polypeptides, comprising:
(a) providing an array comprising a plurality of single-stranded mRNAs (ss-mRNAs), wherein each member of the plurality of ss-mRNAs encodes for a polypeptide; and
(b) translating the plurality of ss-mRNAs to produce a plurality of polypeptides, wherein each member of the plurality of polypeptides remains bound to the corresponding member of the plurality of ss-mRNAs,
thereby generating an array of polypeptides.

3. A method of generating an array of polypeptides, comprising:
(a) providing an array comprising a plurality of clonal spots of single-stranded DNAs (ssDNAs) covalently attached to a surface of the array, wherein some or all of the plurality of ssDNAs encode a polypeptide;
(b) replicating the plurality of ssDNAs to generate a plurality of clonal spots of double-stranded DNAs (dsDNAs);
(c) transcribing the plurality of dsDNAs to produce a plurality of RNA transcripts, wherein each member of the plurality of RNA transcripts remains bound to a corresponding dsDNA-RNA polymerase complex; and
(d) translating the plurality of transcripts to produce a plurality of polypeptides, wherein each member of the plurality of polypeptides remains bound to a corresponding RNA transcript-ribosome complex,
thereby generating an array of polypeptides.

4. A method of claim 3, wherein the transcribing proceeds towards the surface of the array.

5. The method of claim 1, wherein the step of providing the array comprises assembling the plurality of single-stranded nucleic acid sequences on the surface of the array.

6. (canceled)

7. The method of claim 2 wherein the ss-mRNA is attached to the array at its 3′ end.

8. The method of claim 2, wherein the ss-mRNA is attached to the array at its 5′ end.

9. The method of claim 2, wherein the array comprises a plurality of ss-mRNA-DNA-puromycin fusion molecules, wherein each fusion molecule comprises a) an mRNA sequence containing a translation initiation sequence and an open reading frame encoding for a polypeptide attached to the solid substrate by its 5′ end, b) a DNA linker 16 to 40 nucleotides long, and c) a puromycin molecule attached to the 3′ end of the DNA linker.

10. The method of claim 1, wherein the plurality of polypeptides are known polypeptides, unknown polypeptides, random polypeptides, one polypeptide having a variety of mutations, computationally-generated polypeptides, or combinations thereof.

11-13. (canceled)

14. A method of using a polypeptide array made by the method of claim 1, comprising:
(a) contacting the array with one or more ligands; and
(b) determining whether or not the one or more ligands bind to one or more of the plurality of polypeptides on the array; and
(c) optionally, determining which one or more of the plurality of polypeptides on the array is bound by the one or more ligands.

15. A method of using a polypeptide array made by the method of claim 1, comprising:
(a) contacting the array with one or more ligands, wherein the one or more ligands are nucleic acid-barcoded;
(b) ligating the nucleic acid barcode of the ligand to the plurality of ss-mRNA or dsDNA;
(c) determining whether or not the one or more ligands binds to one or more of the plurality of polypeptides on the array by sequencing; and
(d) optionally, determining which one or more of the plurality of polypeptides on the array is bound by the one or more ligands.

16. (canceled)

17. The method of claim 1, wherein the dsDNA or ss-mRNA comprises a nucleic acid barcode prior to the start codon for identifying the polypeptide encoded by the dsDNA or ss-mRNA.

18. The method of claim 1, wherein the dsDNA or ss-mRNA contains a nucleic acid barcode following the coding sequence for identifying the polypeptide.

19. The method of claim 15, wherein the plurality of ligands are selected from the group consisting of antibodies, aptamers, nucleic acids, proteins, peptides, and other small molecule binders.

20. A method of using an array of polypeptides made by the method of claim 1, comprising:
(a) contacting the array with one or more substrates and reaction reagents; and
(b) detecting the presence of activity by one or more of the plurality of polypeptides on the array; and
(c) optionally, determining which one or more of the plurality of polypeptides on the array exhibited activity.

21. A polypeptide array made by the method of claim 1.

22-25. (canceled)

26. The method of claim 1, wherein at least one of the plurality of polypeptides comprises one or more cleavage sites susceptible to cleavage by site-specific proteases.

27-28. (canceled)

29. Polypeptides made by the method of claim 1.

30-31. (canceled)

32. The method of claim 1, wherein the array comprising the plurality of ssDNA sequences is generated by affixing the plurality of ssDNA sequences flanked by adaptor sequences onto an array comprising a lawn of sequences wherein one set of sequences is complementary to one of the flanking adaptor sequences, and the other set of sequences is identical to the other flanking adaptors sequence.

33. (canceled)

34. The method of claim 33, wherein generating the array comprising the plurality of dsDNA on the plurality of beads comprises:
(a) functionalizing the surface of the plurality of beads;
(b) attaching a plurality of oligonucleotide adapters to the functionalized surface of the plurality of beads;
(c) depositing one or more ssDNA variants on each bead; and
(d) amplifying and converting the one or more ssDNA variants into a plurality of dsDNA clones.
